(19) United States
(12) Patent Application Publication
(10) Pub. No.: US 2016/0183805 A1
(43) Pub. Date: Jun. 30, 2016

(54) CALIBRATOR AND CALIBRATION APPARATUS

(71) Applicant: OLYMPUS CORPORATION, Tokyo (JP)

(72) Inventors: Yoshimine KOBAYASHI, Hachioji-Shi (JP); Seiki TORIYAMA, Hino-Shi (JP); Kenji KAMIMURA, Nagaoka-Shi (JP)

(73) Assignee: OLYMPUS CORPORATION, Tokyo (JP)

(21) Appl. No.: 15/064,030

(22) Filed: Mar. 8, 2016

Related U.S. Application Data
(63) Continuation of application No. PCT/JP2014/080531, filed on Nov. 18, 2014.
(60) Provisional application No. 61/914,564, filed on Dec. 11, 2013.

(51) Int. Cl. A61B 5/00

(52) U.S. Cl. CPC A61B 5/0084; A61B 5/0075; A61B 2560/0233

(57) ABSTRACT

A calibrator calibrates optical characteristics of an optical measurement apparatus in water by inserting a measurement probe connected to the optical measurement apparatus into the calibrator. The optical measurement apparatus is configured to perform optical measurement on body tissue. The calibrator includes: an accommodation portion that has an opening at one end thereof and accommodates the water, a distal end of the measurement probe being configured to be inserted into the accommodation portion; and a removing unit that is arranged in the water and is configured to remove a substance adhered to the distal end of the measurement probe inserted into the accommodation portion.

### CROSS-REFERENCE TO RELATED APPLICATIONS

This application is a continuation of PCT international application Ser. No. PCT/JP2014/080531 filed on Nov. 18, 2014 which designates the United States, incorporated herein by reference, and which claims the benefit of priority from U.S. provisional application No 61/914,564 filed on Dec. 11, 2013, incorporated herein by reference.

### BACKGROUND

1. Technical Field

The disclosure relates to a calibrator and a calibration apparatus, which are used in an optical measurement apparatus for measuring optical characteristics of body tissue.

2. Related Art

In recent years, optical measurement apparatuses are known, which irradiate body tissue with illumination light and estimate, based on measurement values of returned light of the illumination light reflected or scattered from the body tissue, characteristics of the body tissue. An optical measurement apparatus is used in combination with an endoscope for observing an organ such as a digestive organ. As such an optical measurement apparatus, an optical measurement apparatus has been proposed, which uses low-coherence enhanced backscattering spectroscopy (LEBS) for detecting characteristics of body tissue by irradiating the body tissue with low-coherence white light of a short spatial coherence length from a distal end of an illumination fiber of a measurement probe and measuring, using a plurality of detection fibers, an intensity distribution of scattered light beams of plural angles (for example, see U.S. Patent Application Publication No. 2009/0009759).

The above mentioned optical measurement apparatus performs a calibration process before starting the detection with respect to the body tissue, in order to ensure detection accuracy. Specifically, a calibrator provided according to a type of calibration is used to perform a calibration process by using a surface light source or a calibration process of white balance that is a reference by using white light.

As one of the calibration processes mentioned above, a probe calibration process is known, which is performed in an environment close to a state in practical use, that is, in an environment simulating a digestive tract mucous membrane surface layer. In this calibration process, optical measurement is performed in a state in which a probe is inserted in a vessel (test tube) containing sterilized water. A technique has been disclosed (for example, see Japanese National Publication of International Patent Application No. 2010-530542), which is for removing bubbles by stirring the liquid, vibrating the probe, or the like, in order to prevent accuracy of the calibration process from decreasing due to the bubbles adhering upon insertion of the probe into the vessel.

### SUMMARY

In some embodiments, a calibrator calibrates optical characteristics of an optical measurement apparatus in water by inserting a measurement probe connected to the optical measurement apparatus into the calibrator. The optical measurement apparatus is configured to perform optical measurement on body tissue. The calibrator includes: an accommodation portion that has an opening at one end thereof and accommodates the water, a distal end of the measurement probe being configured to be inserted into the accommodation portion; and a removing unit that is arranged in the water and is configured to remove a substance adhered to the distal end of the measurement probe inserted into the accommodation portion.

In some embodiments, a calibration apparatus calibrates optical characteristics of an optical measurement apparatus by inserting a measurement probe connected to the optical measurement apparatus into the calibration apparatus. The optical measurement apparatus is configured to perform optical measurement on body tissue. The calibration apparatus includes: a flat field calibrator that includes a surface light source and is configured to calibrate optical characteristics of the measurement probe with respect to uniform light; a white balance calibrator configured to calibrate a white balance as a reference, using white light; a first background calibrator configured to calibrate a background in air; a second background calibrator that is the above-described calibrator and is configured to calibrate a background in water; and a support portion that supports each of the flat field calibrator, the white balance calibrator, the first background calibrator, and the second background calibrator in a standing position.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a block diagram schematically illustrating a configuration of an optical measurement system according to a first embodiment of the present invention;

FIG. 2 is a perspective diagram schematically illustrating a structure of a calibration apparatus of the optical measurement system according to the first embodiment of the present invention;

FIG. 3 is a cross section diagram schematically illustrating a structure of main parts of the calibration apparatus of the optical measurement system according to the first embodiment of the present invention;

FIG. 4 is a perspective diagram schematically illustrating a structure of main parts of the calibration apparatus of the optical measurement system according to the first embodiment of the present invention;

FIG. 5 is a partial cross section diagram schematically illustrating a state in which a measurement probe has been inserted in the calibration apparatus of the optical measurement system according to the first embodiment of the present invention;

FIG. 6 is a diagram illustrating a situation in which the optical measurement system according to the first embodiment of the present invention is used in an endoscopic system;

FIG. 7 is a perspective diagram schematically illustrating a structure of main parts of a calibration apparatus of an optical measurement system according to a modified example of the first embodiment of the present invention;

FIG. 8 is a cross section diagram schematically illustrating a structure of main parts of a calibration apparatus of an optical measurement system according to a second embodiment of the present invention;

FIG. 9 is a perspective diagram schematically illustrating a structure of main parts of the calibration apparatus of the optical measurement system according to the second embodiment of the present invention;

FIG. 10 is a cross section diagram schematically illustrating a structure of main parts of a calibration apparatus of an optical measurement system according to a first modified example of the second embodiment of the present invention;

FIG. 11 is a perspective diagram schematically illustrating a structure of main parts of the calibration apparatus of the optical measurement system according to the first modified example of the second embodiment of the present invention;

FIG. 12 is a cross section diagram schematically illustrating a structure of main parts of a calibration apparatus of an optical measurement system according to a second modified example of the second embodiment of the present invention; and

FIG. 13 is a cross section diagram schematically illustrating a structure of main parts of a calibration apparatus of an optical measurement system according to a third embodiment of the present invention.

### DETAILED DESCRIPTION

Exemplary embodiments of a calibration apparatus according to the present invention will be described below in detail with reference to the drawings. The present invention is not limited by the embodiments. The same reference signs are used to designate the same elements throughout the drawings. Further, the drawings are schematic, and it is to be noted that the relation between the thickness and width of each element and the ratios among the respective elements are different from the actual. Further, a portion is included, which has different size relations and ratios among the drawings.

### First Embodiment

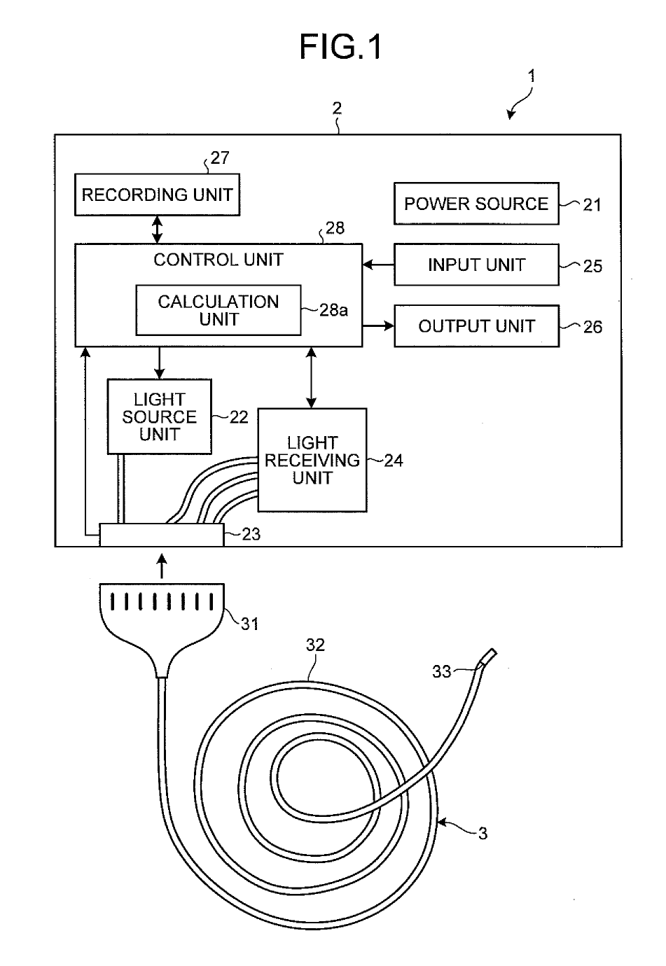

FIG. 1 is a block diagram that schematically illustrates a configuration of an optical measurement system according to a first embodiment of the present invention. An optical measurement system 1 illustrated in FIG. 1 includes: an optical measurement apparatus 2 that performs optical measurement on a measurement target as a scatterer which is an object to be measured such as body tissue, and that detects properties (characteristics) of the measurement target; and a measurement probe 3 that is detachable from the optical measurement apparatus 2, is inserted into a subject, and is for measurement.

First, the optical measurement apparatus 2 is described. The optical measurement apparatus 2 includes a power source 21, a light source unit 22, a connection unit 23, a light receiving unit 24, an input unit 25, an output unit 26, a recording unit 27, and a control unit 28. The power source 21 supplies electric power to each element of the optical measurement apparatus 2.

The light source unit 22 is realized by using an incoherent light source such as a white light emitting diode (LED), a xenon lamp, a tungsten lamp, and a halogen lamp, and as necessary, one or more lenses, for example, a condenser lens, a collimator lens, or the like. The light source unit 22 outputs to the measurement probe 3 via the connection unit 23, incoherent light having at least one spectral component for irradiating the measurement target.

The connection unit 23 detachably connects a connector portion 31 of the measurement probe 3 to the optical measurement apparatus 2. The connection unit 23 outputs light generated by the light source unit 22 to the measurement probe 3 and outputs, to the light receiving unit 24, returned light of illumination light, which has been emitted from the measurement probe 3 and reflected and/or scattered from the measurement target. The connection unit 23 outputs, to the control unit 28, information related to existence or non-existence of connection of the measurement probe 3.

The light receiving unit 24 receives and measures the returned light of the illumination light, which is the illumination light emitted from the measurement probe 3 and reflected and/or scattered from the measurement target. The light receiving unit 24 is realized by sing a plurality of spectrometers, light receiving sensors, or the like. Specifically, the light receiving unit 24 is provided with the spectrometers, according to the number of later described light receiving fibers of the measurement probe 3. The light receiving unit 24 measures a spectral component and an intensity distribution of scattered light entering from the measurement probe 3 and performs measurement of each wavelength. The light receiving unit 24 outputs results of the measurement to the control unit 28.

The input unit 25 is realized by using a push-type switch, a touch panel, or the like, receives input of an instruction signal instructing activation of the optical measurement apparatus 2 or an instruction signal instructing various other operations, and outputs the received input to the control unit 28.

The output unit 26 is realized by using a display of a liquid crystal or organic electroluminescence (EL), and a speaker or the like, and outputs information related to various processes in the optical measurement apparatus 2. Further, the output unit 26 displays, under control by the control unit 28, numerical values such as intensities of light received by the light receiving unit 24 (characteristic values calculated by a calculation unit 28a described later).

The recording unit 27 is realized by using a volatile memory or a non-volatile memory, and records therein various programs for operating the optical measurement apparatus 2 and various data and various parameters used in an optical measurement process. The recording unit 27 temporarily records therein information during a process by the optical measurement apparatus 2. Further, the recording unit 27 records therein results of the measurement by the optical measurement apparatus 2 in association with the subject, which is the measurement target. The recording unit 27 may be configured by using a memory card or the like inserted from outside of the optical measurement apparatus 2.

The control unit 28 is configured using a central processing unit (CPU) or the like. The control unit 28 controls operations of a process by each unit of the optical measurement apparatus 2. The control unit 28 controls the operations of the optical measurement apparatus 2 by performing transfer or the like of instruction information and data corresponding to each unit of the optical measurement apparatus 2. The control unit 28 records the results of the measurement by the light receiving unit 24 in the recording unit 27. The control unit 28 has the calculation unit 28a.

Based on the results of the measurement by the light receiving unit 24, the calculation unit 28a performs a plurality of calculation processes and calculates characteristic values related to the characteristics of the measurement target. The types of these characteristic values are set according to the instruction signal received by the input unit 25, for example.

Next, the measurement probe 3 is explained. The measurement probe 3 is realized by arranging a plurality of optical fibers inside thereof. Specifically, the measurement probe 3 is realized by using an illumination fiber that irradiates the measurement target with the illumination light and the plurality of light receiving fibers into which the returned light of the illumination light reflected and/or scattered from the measurement target enter at different angles. The measurement probe 3 has the connector portion 31 detachably connected to the connection unit 23 of the optical measurement apparatus 2, a flexible portion 32 having a flexibility, and a distal end portion 33 which outputs the illumination light supplied from the light source unit 22 and receives the returned light from the measurement target. The illumination fiber and the light receiving fibers are each configured using a step index single core fiber.

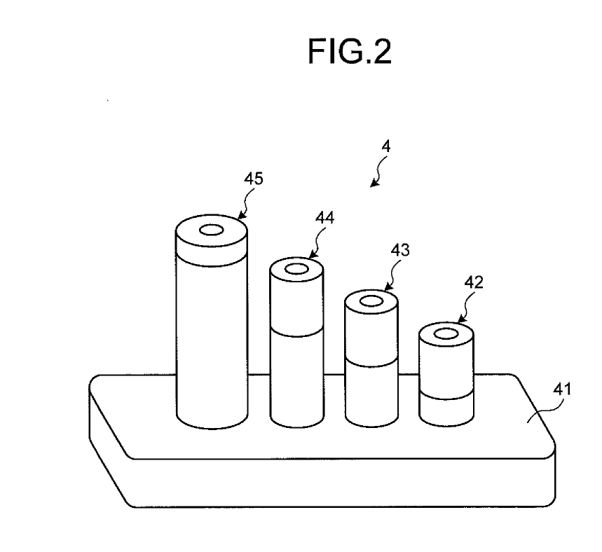

Next, a calibration apparatus for calibrating the measurement probe 3 is described with reference to FIGS. 2 to 5. FIG. 2 is a perspective diagram that schematically illustrates a configuration of a calibration apparatus of the optical measurement system according to the first embodiment of the present invention. A calibration apparatus 4 illustrated in FIG. 2 is formed of a plurality of calibrators provided according to a type of calibration and a support portion 41 that supports the plurality of calibrators in their standing state.

The plurality of calibrators include a flat field calibrator 42 for calibrating optical characteristics of the measurement probe 3 with respect to uniform light, a white balance calibrator 43 for calibrating a white balance that is a reference, a first background calibrator 44 for calibrating a background in the air, and a second background calibrator 45 for calibrating a background in water. Each of these calibrators is approximately columnar and provided with an accommodation hole for inserting and accommodating the distal end portion 33 of the measurement probe 3.

By performing optical measurement by the measurement probe 3 using each of the above mentioned calibrators, calibration factors and calibration values are calculated by the calculation unit 28a. Specifically, the distal end portion 33 of the measurement probe 3 is inserted in the accommodation hole of the flat field calibrator 42 for example, and light for calibration is received by the light receiving fibers and output to the light receiving unit 24. Measurement values obtained from the measurement target such as the body tissue or the like are corrected by the calculation unit 28a based on the above mentioned calibration factors and calibration values and output as accurate optical measurement values.

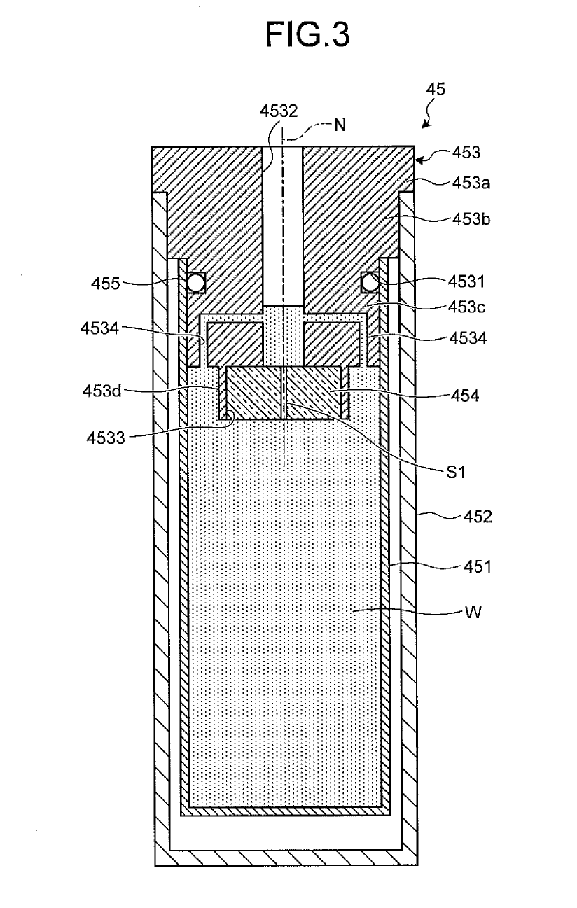

FIG. 3 is a cross section diagram that schematically illustrates a structure of main parts of the calibration apparatus of the optical measurement system according to the first embodiment of the present invention and a cross section thereof is a plane perpendicular to a longitudinal direction of the calibration apparatus. FIG. 3 is a diagram that illustrates the second background calibrator 45 illustrated in FIG. 2.

The second background calibrator 45 includes: a first accommodation portion 451 that has an opening at one end thereof and accommodates water W, which is a background; a second accommodation portion 452 that has an opening at one end thereof and accommodates the first accommodation portion 451; a sealing member 453 that seals each of the openings of the first accommodation portion 451 and the second accommodation portion 452; and a removing member 454 that removes a substance adhered to the distal end of the measurement probe 3, by the measurement probe 3 being inserted therethrough after the removing member 454 comes into contact with the distal end of the measurement probe 3 upon accommodation of the distal end portion 33 of the measurement probe 3 in the first accommodation portion 451. Between the first accommodation portion 451 and the sealing member 453, an O-ring 455 is provided, which fixes between the first accommodation portion 451 and the sealing member 453 and prevents the water W accommodated in the first accommodation portion 451 from leaking outside from between the first accommodation portion 451 and the sealing member 453.

The first accommodation portion 451 is formed of a transparent resin, glass, or the like, for example. The second accommodation portion 452 is formed of a light shielding material (for example, a resin or a metal) or is formed by at least a surface thereof being covered by a light shielding resin, metal, or the like, such that light does not enter inside. Further, in the vicinity of the opening of the second accommodation portion 452, a concavo-convex shape for being screwed with the sealing member 453 is formed.

The sealing member 453 includes: a tip end portion 453a that is provided at one end thereof and has an outer peripheral diameter approximately equal to an outer peripheral diameter of the second accommodation portion 452; a middle portion 453b that extends from an end of the tip end portion 453a and has an outer peripheral diameter approximately equal to an inner peripheral diameter of the second accommodation portion 452 (a diameter of the opening); and a base end portion 453c that extends from an end of the middle portion 453b different from an end thereof closer to the tip end portion 453a and has an outer peripheral diameter approximately equal to an inner peripheral diameter of the first accommodation portion 451 (a diameter of the opening).

On an outer periphery of the middle portion 453b, a concavo-convex shape for being screwed with the second accommodation portion 452 is formed.

Along an outer periphery of the base end portion 453c, a groove portion 4531 for accommodating the O-ring 455 is formed. A notched depth of the groove portion 4531 is a little smaller than a diameter of a wire material forming the O-ring 455.

The sealing member 453 has a through hole 4532 formed therein, which penetrates in a direction parallel to a direction in which the tip end portion 453a, the middle portion 453b, and the base end portion 453c extend one after another. A diameter of an opening of the through hole 4532 is a little larger than a diameter of the measurement probe 3.

The base end portion 453c has a cylindrical portion 453d that extends, cylindrically shaped, from an end different from an end closer to the middle portion 453b and has an outer peripheral diameter smaller than the diameter of the base end portion 453c. A diameter of an opening of a hollow portion 4533 of the cylindrical portion 453d is larger than the diameter of the opening of the through hole 4532.

In the base end portion 453c, two communication holes 4534 are formed for connecting first opening parts provided on an outer periphery side of an area where the cylindrical portion 453d is formed (an area where the removing member 454 is arranged) and provided on a surface from which the cylindrical portion 453d extends, with second opening parts formed on a wall surface of the through hole 4532.

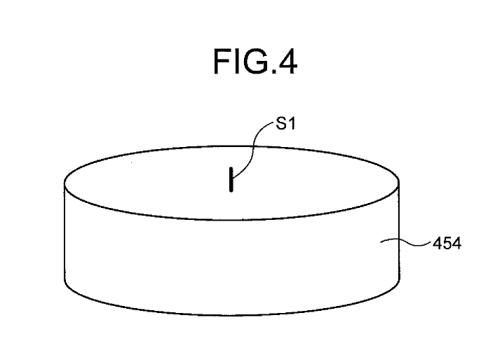

FIG. 4 is a perspective diagram that schematically illustrates main parts of the calibration apparatus of the optical measurement system according to the first embodiment and illustrates a structure of the removing member 454. The removing member 454 is formed by using an elastically deformable material, for example a resin (for example a sponge made of a synthetic resin) or rubber, is column shaped, and has an outer peripheral diameter that is equal to or a little larger than the diameter of the opening of the hollow portion 4533. The removing member 454 has a slit S1 formed therein such that the slit S1 intersects with a central axis thereof and the slit S1 penetrates through the removing member 454 in a direction of the central axis. A formed width of the slit S1 (a width in a direction perpendicular to the central axis of the removing member 454) is larger than the diameter of the measurement probe 3 (the distal end portion 33) and designed to be of a size through which the measurement probe 3 is insertable.

The removing member 454 is held by the sealing member 453 by being accommodated in or pressed into the hollow portion 4533. The removing member 454 is arranged at a position through which a central axis N of the through hole 4532 passes and in the water W. When the removing member 454 is arranged in the hollow portion 4533, the slit S1 is preferably arranged on the central axis N of the through hole 4532. Further, at an end of the cylindrical portion 453d, a claw that protrudes towards an inner periphery may be provided to hold the removing member 454.

In the second background calibrator 45, the first accommodation portion 451 accommodates the water W of an amount that covers the openings on the through hole 4532 side of the communication holes 4534 when the sealing member 453 is attached to the first accommodation portion 451. Accordingly, when the sealing member 453 is attached to the first accommodation portion 451, the removing member 454 is positioned in the water W. The amount of the accommodated water W is not limited to this as long as the amount is such that when the sealing member 453 is attached to the first accommodation portion 451, the removing member 454 is positioned in the water W and when the measurement probe 3 is inserted, the water does not leak out from the sealing member 453.

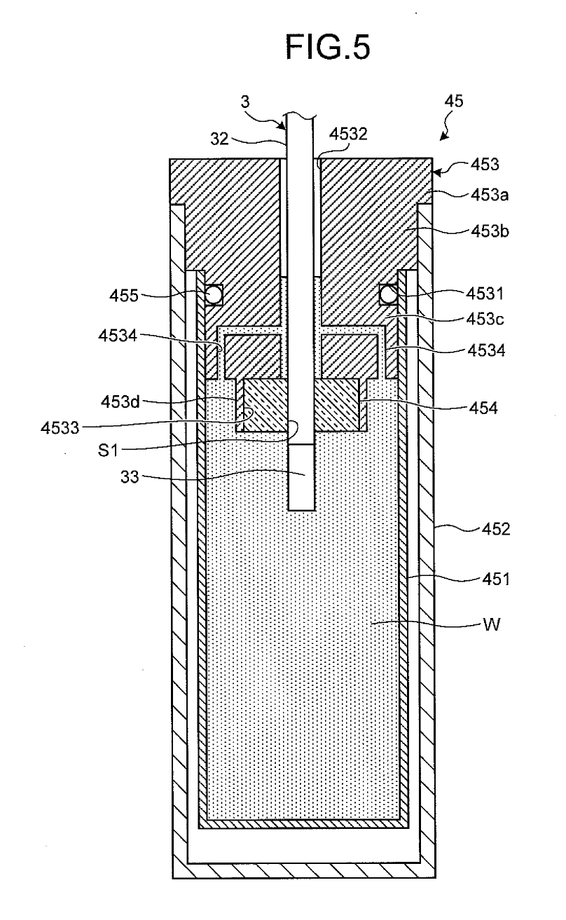

FIG. 5 is a partial cross section diagram that schematically illustrates a state in which a measurement probe has been inserted in the calibration apparatus of the optical measurement system according to the first embodiment of the present invention. When a background in water is calibrated, the measurement probe 3 is inserted in the second background calibrator 45 and optical measurement is performed in a state in which the distal end portion 33 is positioned in the water W.

When the measurement probe 3 is inserted in the second background calibrator 45, the distal end portion 33 enters into the water W after being inserted inside the sealing member 453 from the through hole. When this is done, bubbles may adhere to the distal end of the distal end portion 33. If bubbles adhere thereto, the distal end portion 33 moves in the water W with the bubbles being adhered to the distal end thereof. Further, by insertion of the measurement probe 3, the water W flows towards the through hole 4532 via the communication holes 4534 and the slit S1.

Thereafter, the distal end portion 33 comes into contact with the removing member 454. When this happens, the bubbles are removed from the distal end portion 33 after being interposed between an end face of the distal end portion 33 and an end face of the removing member 454, for example.

After coming into contact with the removing member 454, the distal end portion 33 is pressed into the slit S1 and inserted to a specified position in the first accommodation portion 451. Herein, on a lateral face of the measurement probe 3, an insertion position of the measurement probe 3 with respect to each calibrator is recorded. A user is able to insert the measurement probe 3 to the specified position by inserting the measurement probe 3 into the calibrator while checking the insertion position thereof.

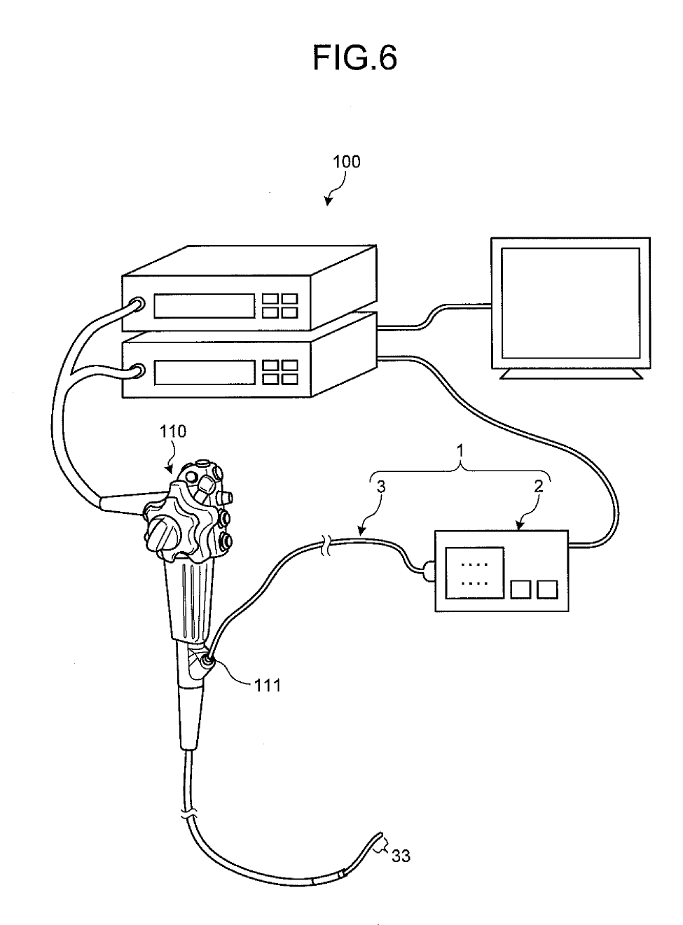

As illustrated in FIG. 6, in the above described optical measurement system 1, after a calibration process of the optical measurement apparatus 2 using the above described calibration apparatus 4 is performed, the measurement probe 3 is inserted in the subject via a treatment tool channel 111 provided in an endoscopic device 110 (endoscope) of an endoscopic system 100, the illumination fiber irradiates the measurement target with the illumination light, and the plurality of light receiving fibers respectively receive, at different scattering angles, the returned light of the illumination light, which has been reflected and/or scattered from the measurement target, and propagate the received returned light to the light receiving unit 24 of the optical measurement apparatus 2. Thereafter, the calculation unit, 28a calculates the characteristic values of the characteristics of the measurement target, based on the results of the measurement by the light receiving unit 24.

According to the above described first embodiment of the present invention, in the calibration apparatus 4 for performing calibration of the optical measurement apparatus 2 by inserting the measurement probe 3, the second background calibrator 45 has the removing member 454 that contacts the measurement probe 3 in the water W, which is the background, and removes the bubbles adhered to the distal end portion 33, and thus a simple structure and downsizing can be realized.

According to the above described first embodiment, shapes of the inner periphery of the cylindrical portion 453d and the outer periphery of the removing member 454 have been described as being circular, but the shapes may be angular, for example. By being angular, the removing member 454 is preventable from sliding and rotating along an inner wall surface of the cylindrical portion 453d, and a direction of the slit S1 is infallibly fixable.

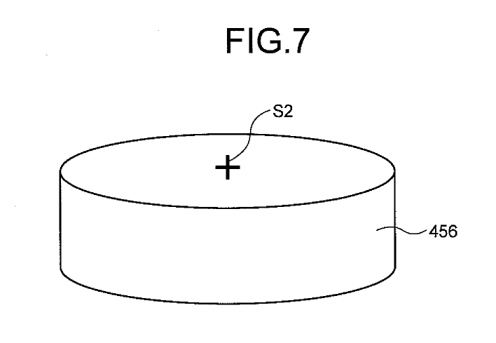

Modified Example of First Embodiment FIG. 7 is a perspective diagram that schematically illustrates a structure of main parts of a calibration apparatus of an optical measurement system according to a modified example of the first embodiment. The removing member 454 has been described as being formed with the slit S1 by being cut in one direction, but as illustrated in FIG. 7, a removing member 456 may be formed with a slit S2 that has been cut to be cross-shaped. The removing member 456 has a structure similar to that of the above described removing member 454 except for the mode of forming the slit S2.

According to the modified example, because the slit S2 that has been cut to be cross-shaped is formed, compared with the above described slit S1 formed by being cut in one direction, insertion of the measurement probe 3 is possible with a small load and the adhered bubbles are infallibly removable.

### Second Embodiment

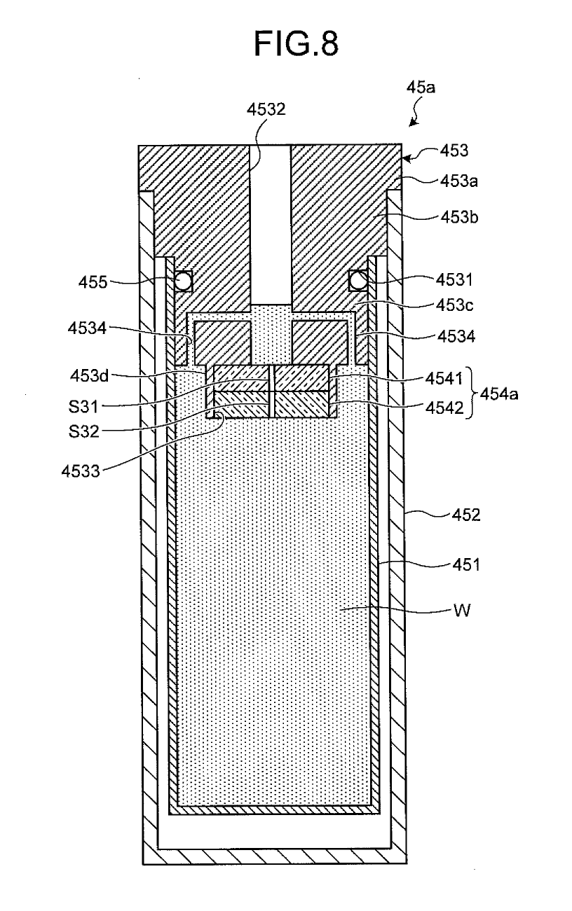

Next, a second embodiment of the present invention will be described. FIG. 8 is a cross section diagram that schematically illustrates a structure of main parts of a calibration apparatus of an optical measurement system according to the second embodiment of the present invention and a cross section thereof is a plane perpendicular to a longitudinal direction of the calibration apparatus. Elements which are the same as those described above are denoted by the same reference signs. In the above described first embodiment, the removing member 454 has been described as being formed of a single member, but a second background calibrator 45a according to the second embodiment has a removing member 454a layered of a plurality of members.

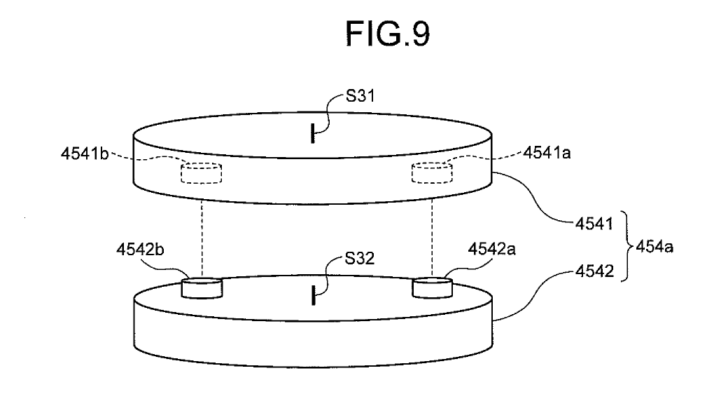

FIG. 9 is a perspective diagram that schematically illustrates a structure of main parts of the calibration apparatus of the optical measurement system according to the second embodiment and illustrates a structure of the removing member 454a. The removing member 454a is formed of an elastically deformable material, for example, a resin (for example, a sponge made of a synthetic resin) or rubber and has a first removing member 4541 that is column shaped and formed with a slit S31 to intersect with a central axis thereof and a second removing member 4542 that is approximately column shaped and formed with a slit S32 to intersect with the central axis, and the first removing member 4541 and the second removing member 4542 are layered over each other. The slits S31 and S32 are formed by being cut in one direction to respectively penetrate through the first removing member 4541 and the second removing member 4542.

The first removing member 4541 has concave portions 4541a and 4541b, which are formed on one side thereof, have circular openings, and are concave shaped. The second removing member 4542 has convex portions 4542a and 4542b, which are formed on one side thereof and are convex shaped to protrude columnarly. Diameters of the openings of the concave portions 4541a and 4541b are equal to diameters of the convex portions 4542a and 4542b in a direction perpendicular to a protrusion direction thereof. The concave portions 4541a and 4541b and the convex portions 4542a and 4542b are provided at positions symmetrical with respect to the respective central axes of the first removing member 4541 and the second removing member 4542.

When the first removing member 4541 and the second removing member 4542 are layered over each other, the convex portion 4542a fits into the concave portion 4541a and the convex portion 4542b fits into the concave portion 4541b. Accordingly, a layering position of the second removing member 4542 with respect to the first removing member 4541 is fixed, and directions of the slit S31 and slit S32 are able to be prescribed.

When the measurement probe 3 is inserted in the second background calibrator 45a, the distal end portion 33 enters into the water W after being inserted inside the sealing member 453 from the through hole 4532. When this is done, bubbles may adhere to the distal end of the distal end portion 33. If bubbles adhere thereto, the distal end portion 33 moves in the water W with the bubbles being adhered to the distal end thereof.

Thereafter, the distal end portion 33 comes into contact with a top surface of the first removing member 4541. When this happens, the bubbles are removed from the distal end portion 33 after being interposed between the end face of the distal end portion 33 and the top surface of the first removing member 4541, for example.

The distal end portion 33 is pressed into the slit S31 after coming into contact with the top surface of the first removing member 4541. Thereafter, the distal end portion 33 comes into contact with a top surface of the second removing member 4542, is pressed into the slit S32, and is inserted to a specified position in the first accommodation portion 451. In this insertion operation, with respect to the removing member 454a, the distal end portion 33 stepwisely comes into contact with the top surfaces of the first removing member 4541 and the second removing member 4542. Accordingly, even if the bubbles are not removable by the contact with the first removing member 4541, the bubbles are removable by the contact with the second removing member 4542.

According to the above described second embodiment of the present invention, in the calibration apparatus 4 for performing calibration of the optical measurement apparatus 2 by inserting the measurement probe 3, the second background calibrator 45a has the removing member 454a, which is formed of the first removing member 4541 and the second removing member 4542 layered over each other, and which removes, by contacting the measurement probe 3 in the water W that is the background, the bubbles adhered to the distal end portion 33, and thus a simple structure and downsizing can be realized.

### First Modified Example of Second Embodiment

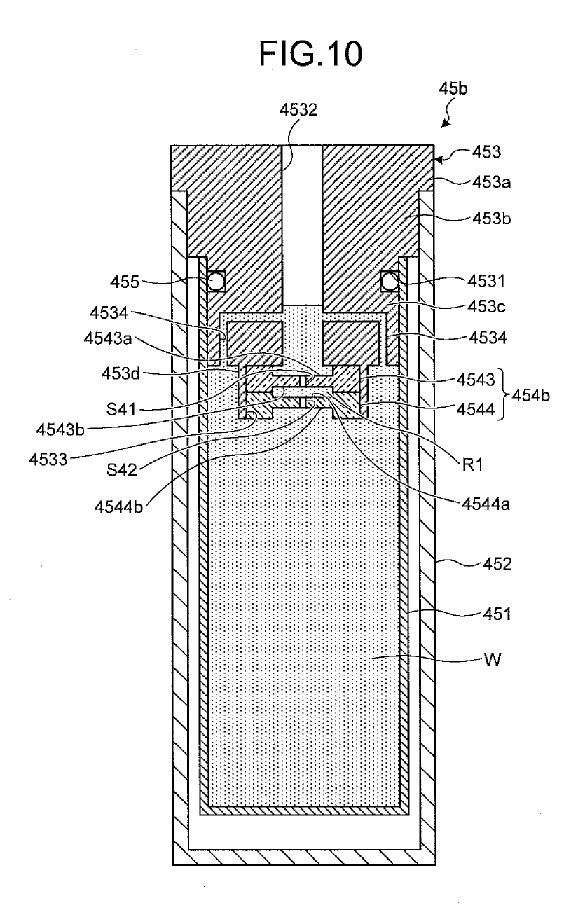

FIG. 10 is a cross section diagram that schematically illustrates a structure of main parts of a calibration apparatus of an optical measurement system according to a first modified example of the second embodiment and a cross section thereof is a plane perpendicular to a longitudinal direction of the calibration apparatus. A second background calibrator 45b according to the first modified example is provided with a removing member 454b, instead of the removing member 454a of the above described second background calibrator 45a.

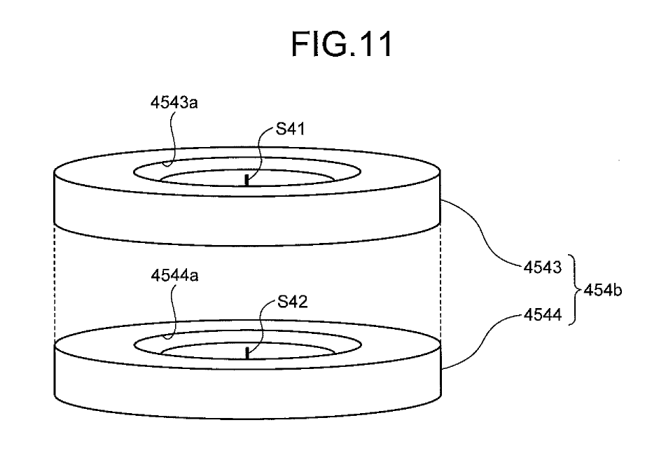

FIG. 11 is a perspective diagram that schematically illustrates a structure of main parts of the calibration apparatus of the optical measurement system according to the first modified example of the second embodiment. The removing member 454b has a first removing member 4543 that is approximately column shaped, and a second removing member 4544 that is approximately column shaped, and the first removing member 4543 and the second removing member 4544 are layered over each other.

The first removing member 4543 is formed with a first hole portion 4543a that forms a columnar hollow space from one side of the first removing member 4543, and a second hole portion 4543b that forms a columnar hollow space from the other side of the first removing member 4543.

The second removing member 4544 is formed with a first hole portion 4544a that forms a columnar hollow space from one side of the second removing member 4544, and a second hole portion 4544b that forms a columnar hollow space from the other side of the second removing member 4544.

The first removing member 4543 and the second removing member 4544 are respectively formed with slits S41 and S42 that intersect with central axes thereof. The slits S41 and S42 are formed by being cut in one direction to respectively penetrate through the first removing member 4543 and the second removing member 4544.

When the first removing member 4543 and the second removing member 4544 are layered over each other, the hole portions (in FIGS. 10 and 11, the second hole portion 4543b and the first hole portion 4544a) provided on mutually facing sides communicate with each other to form a room After the bubbles that are not removable by the first removing member 4543 are removed by the second removing member 4544, the removed bubbles are held within the room R1. Accordingly, deformation of the first removing member 4543 and second removing member 4544 is preventable, which is caused by the removed bubbles stagnating in the slits or between the first removing member 4543 and the second removing member 4544.

Further, according to the first modified example, because the first removing member 4543 and the second removing member 4544 are formable using the same member, productivity related to manufacture of each member can be improved.

### Second Modified Example of Second Embodiment

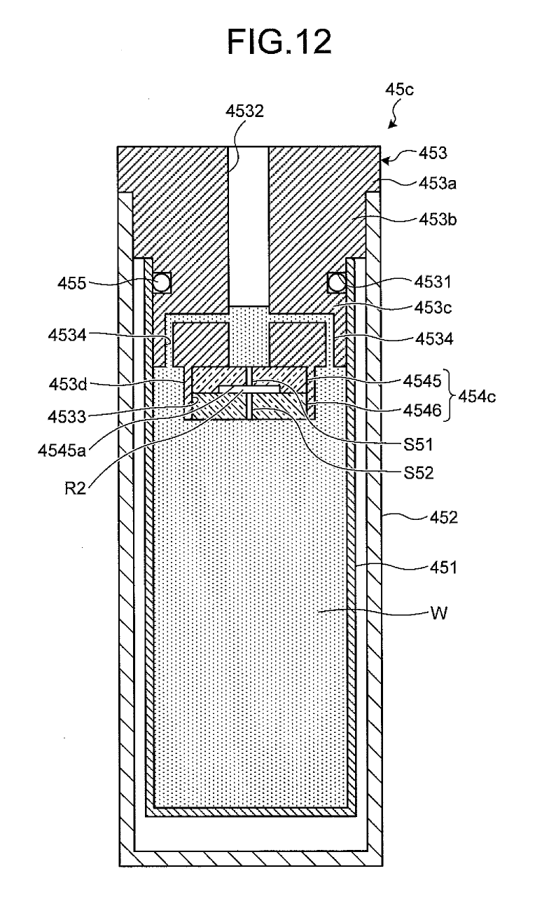

FIG. 12 is a cross section diagram that schematically illustrates a structure of main parts of a calibration apparatus of an optical measurement system according to a second modified example of the second embodiment. In the above described first modified example, formation of the room is applicable as long as the formation is at least between the first removing member 4543 and the second removing member 4544. Therefore, as illustrated in FIG. 12, a hole portion may be formed on any one of a first removing member and a second removing member.

In a second background calibrator 45c illustrated in FIG. 12, a removing member 454c has a first removing member 4545 that is approximately column shaped and a second removing member 4546 that is approximately column shaped, and the first removing member 4545 and the second removing member 4546 are layered over each other.

The first removing member 4545 is formed with a hole portion 4545a that forms a columnar hollow space from one side of the first removing member 4545.

The first removing member 4545 and the second removing member 4546 are respectively formed with slits S51 and S52 that intersect with central axes thereof. The slits S51 and S52 are formed by being cut in one direction to respectively penetrate through the first removing member 4545 and the second removing member 4546.

In layering the first removing member 4545 and the second removing member 4546 over each other, when a side on which the hole portion 4545a is formed is placed to face the second removing member 4546, the hole portion 4545a is blocked by a top surface of the second removing member 4546 to form a room R2. When the bubbles that are not removable by the first removing member 4545 are removed by the second removing member 4546, the removed bubbles are held within the room R2. Accordingly, deformation of the first removing member 4545 and second removing member 4546 are preventable, which is caused by the removed bubbles stagnating in the slits or between the first removing member 4545 and the second removing member 4546.

In the above described first and second modified examples, concave portions and convex portions like in the second embodiment (concave portions 4541a and 4541b and convex portions 4542a and 4542b) may be formed. By forming the concave portions and convex portions, positions of the slits can be defined.

In the above described second embodiment and the first and second modified examples, the directions in which the respective slits in openings in the first and second removing members are formed (directions of the cuts) may be the same (parallel) or may intersect each other, when the removing members are viewed from above. In the second embodiment, in order to be able to discharge the bubbles removed by the second removing member to outside of the removing member 454a, the direction in which the slits are formed preferably do not intersect each other.

### Third Embodiment

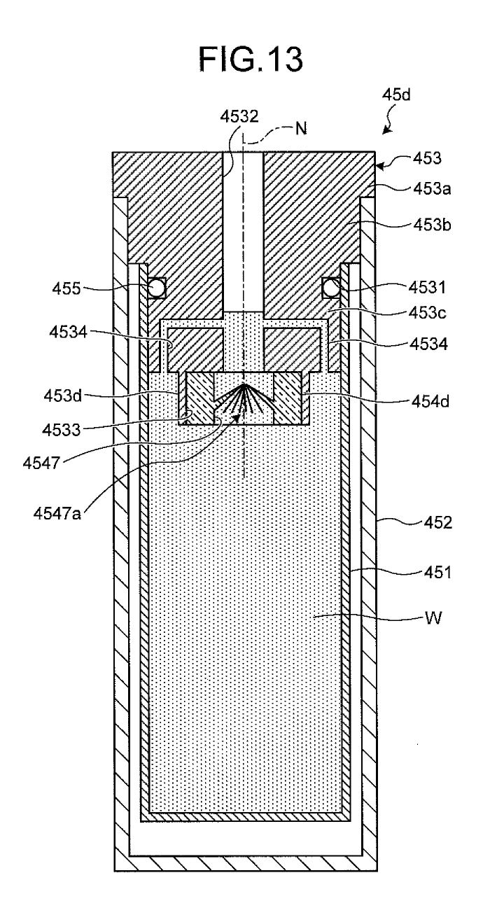

Next, a third embodiment of the present invention will be described. FIG. 13 is a cross section diagram that schematically illustrates a structure of main parts of a calibration apparatus of an optical measurement system according to the third embodiment of the present invention and a cross section thereof is a plane perpendicular to a longitudinal direction of the calibration apparatus. Elements which are the same as those described above are denoted by the same reference signs. In the above described first and second embodiments, the removing members have been described as being formed with the slits, but a second background calibrator 45d according to the third embodiment has a removing member 454d having a brush-shaped removing member.

The removing member 454d illustrated in FIG. 13 is formed of a cylindrical member and a diameter of an outer periphery thereof is equal to or a little larger than a diameter of an inner periphery of the above described hollow portion 4533. The removing member 454d has a hollow portion 4547, which is a cylindrical hollow space. The hollow portion 4547 is formed with a columnar hollow space and a diameter of the hollow space is equal to or a little larger than the diameter of the through hole 4532.

The hollow portion 4547 of the removing member 454d is provided with a contact portion 4547a formed of a plurality of needle-shaped members, which protrude diagonally upward towards a central axis thereof from an inner wall surface thereof and are elastically deformable. In the third embodiment, the central axis of the hollow portion 4547 coincides with the central axis N of the through hole 4543.

When the measurement probe 3 is inserted in the second background calibrator 45d, the distal end portion 33 enters into the water W after being inserted inside the sealing member 453 from the through hole 4532. When this is done, bubbles may adhere to the distal end of the distal end portion 33. If bubbles adhere thereto, the distal end portion 33 moves in the water W with the bubbles being adhered to the distal end thereof.

Thereafter, the distal end portion 33 moves into the removing member 454d to contact (abut) the needle-shaped member of the contact portion 4547a. When this happens, the bubbles are removed from the distal end portion 33 by the needle-shaped member contacting a distal end surface thereof while being elastically deformed, for example.

According to the above described third embodiment of the present invention, in the calibration apparatus 4 for performing calibration of the optical measurement apparatus 2 by inserting the measurement probe 3, the second background calibrator 45d has the removing member 454d that has the plurality of needle-shaped members, which contact the measurement probe 3 in the water W as the background, and remove the bubbles adhered to the distal end portion 33, and thus a simple structure and downsizing can be realized.

In order to suppress increase of manufacture cost, the removing member 454d according to the third embodiment is preferably formed of the same material, but anything is applicable as long as at least the contact portion 4547a is formed using an elastically deformable material, for example, a resin (for example, a sponge made of a synthetic resin) or rubber.

In the above described first to third embodiments, in order to check a position of the distal end of the measurement probe 3 in the first accommodation portion 451, the second accommodation portion 452 may be provided with an openable window. This window is, for example, provided in the vicinity of a bottom portion of the removing member 454 of the second background calibrator 45 and allows the distal end of the measurement probe 3 that has passed the removing member 454 to be observed. Accordingly, whether or not the bubbles remain on the measurement probe 3 that has passed the removing member 454 is able to be checked, and the bubbles adhered to the measurement probe 3 are able to be removed more infallibly.

In the above described first to third embodiments, the water W has been described as flowing towards the through hole 4532 via the communication holes 4534 provided in the sealing member 453, but a structure without the communication holes 4534 is applicable, as long as the water W is able to flow towards the through hole 4532 via the slits.

Further, in the above described first to third embodiments, the removing members have been described as removing the bubbles adhered on the distal end of the measurement probe 3, but not being limited to bubbles, adhered substance like dust is removable. By removing such an adhered substance, a calibration process that is more accurate is possible.

According to some embodiments, it is possible to achieve simply-structured and small-sized calibrator and calibration apparatus.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A calibrator for calibrating optical characteristics of an optical measurement apparatus in water by inserting a measurement probe connected to the optical measurement apparatus into the calibrator, the optical measurement apparatus being configured to perform optical measurement on body tissue, the calibrator comprising:
an accommodation portion that has an opening at one end thereof and accommodates the water, a distal end of the measurement probe being configured to be inserted into the accommodation portion; and
a removing unit that is arranged in the water and is configured to remove a substance adhered to the distal end of the measurement probe inserted into the accommodation portion.

2. The calibrator according to claim 1, wherein
the removing unit has an elastically deformable and approximately columnar shape, and
the removing unit includes a slit that penetrates in a direction of inserting the measurement probe.

3. The calibrator according to claim 1, wherein
the removing unit includes elastically deformable first and second members that are layered over one another, and
each of the first and second members includes a slit that penetrates in a direction of inserting the measurement probe.

4. The calibrator according to claim 3, wherein at least one of the first and second members includes a hole portion having a columnar hollow space that faces the other one of the first and second members.

5. The calibrator according to claim 1, wherein the removing unit includes:
a hollow portion having a columnar hollow space; and
an elastically deformable needle-shaped member that extends from an inner wall surface of the hollow portion towards a center.

6. The calibrator according to claim 1, further comprising a sealing member configured to seal the opening, the sealing member comprising:
a through hole through which the measurement probe is configured to be inserted; and
a communication hole for connecting a first opening part on the sealing member on an outer periphery side of an area where the removing unit is arranged, with a second opening part on a wall surface of the through hole.

7. A calibration apparatus for calibrating optical characteristics of an optical measurement apparatus by inserting a measurement probe connected to the optical measurement apparatus into the calibration apparatus, the optical measurement apparatus being configured to perform optical measurement on body tissue, the calibration apparatus comprising:
a flat field calibrator that includes a surface light source and is configured to calibrate optical characteristics of the measurement probe with respect to uniform light;
a white balance calibrator configured to calibrate a white balance as a reference, using white light;
a first background calibrator configured to calibrate a background in air;
a second background calibrator that is the calibrator of claim I and is configured to calibrate a background in water; and
a support portion that supports each of the flat field calibrator, the white balance calibrator, the first background calibrator, and the second background calibrator in a standing position.
